(19) United States
(12) Patent Application Publication
(10) Pub. No.: US 2024/0254453 A1
(43) Pub. Date: Aug. 1, 2024

(54) CELL CULTURE MEDIUM AND SUPPLEMENTS FOR CORNEAL AND SKIN CELL CULTURE

(71) Applicant: 3D BIO-TISSUES LIMITED, Newcastle Upon Tyne (GB)

(72) Inventors: Che John CONNON, Newcastle Upon Tyne (GB); Ricardo GOUVEIA, Newcastle Upon Tyne (GB)

(21) Appl. No.: 18/578,286

(22) PCT Filed: Jul. 12, 2022
(86) PCT No.: PCT/GB2022/051811

§ 371 Date: Jan. 10, 2024

(30) Foreign Application Priority Data
Jul. 12, 2021 (GB) 2110035.9

(51) Int. Cl. C12N 5/071; C12M 3/00

(52) U.S. Cl. CPC C12N 5/0698; C12M 21/08; C12N 2500/25; C12N 2500/50; C12N 2500/90; C12N 2513/00; C12N 2533/30; C12N 2533/70

(57) ABSTRACT

The present invention provides novel cell culture media and supplements for use in serum-free or reduced-serum cell culture. Corresponding methods and uses are also provided. The novel cell culture media and supplements are particularly beneficial when used in tissue engineering, for example during the in vitro cell culture of corneal cells and/or skin cells.

The present invention provides novel cell culture media and supplements for use in serum-free or reduced-serum cell culture. Corresponding methods and uses are also provided. The novel cell culture media and supplements are particularly beneficial when used in tissue engineering, for example during the in vitro cell culture of corneal cells and/or skin cells.

### BACKGROUND

The field of tissue engineering has made tremendous progress in the past decade and is still evolving. It is not only now possible to control cells and their environments more precisely but also to engineer living tissues and organs of increasing complexities for potential clinical and commercial use. Several commercial and clinical avenues are being pursued in this field, including the creation of tissue, such as corneal or skin substitutes, for use in therapy or other industrial applications such as lab grown leather.

The creation of corneal stromal equivalents, particularly using natural components, typifies many of the challenges in tissue engineering. A suitable corneal stromal substitute needs to be biocompatible, mechanically stable and optically transparent, as well as capable of resisting contraction and proteolytic degradation before and after tissue transplantation. More recently, corneal stromal tissues have been engineered in vitro by means of bottom-up strategies, using the cells themselves to generate the extracellular matrix so as to recreate the well-defined, 3D microarchitecture of the native cornea.

The skin is responsible for several important physiological functions and has enormous clinical significance in wound healing. Tissue engineered substitutes may be used in patients suffering from skin injuries to support regeneration of the epidermis, dermis, or both. Skin substitutes are also gaining traction in the cosmetics and pharmaceutical industries as alternatives to animal models for product testing. Recent biomedical advances, ranging from cellular-level therapies such as mesenchymal stem cell or growth factor delivery, to large-scale biofabrication techniques including 3D printing, have enabled the implementation of unique strategies and novel biomaterials to recapitulate the biological, architectural, and functional complexity of native skin. In addition, using skin tissue engineering to generate commercial revenue for example from lab-grown leather, is also gaining traction.

One of the current challenges facing this field is the limited availability of cell culture resources that can be used on a commercial scale and/or in a clinical setting.

There is a need for improved cell culture media and supplements for use in tissue engineering, including engineering of corneal and skin tissue.

### BRIEF SUMMARY OF THE DISCLOSURE

The invention is based on the surprising finding that specific macromolecular crowding (MMC) agents are useful supplements during the culture of corneal cells and/or skin cells. These agents can surprisingly promote cell proliferation, even when no serum or reduced-serum is used. These agents can therefore advantageously be used as an alternative supplement to serum during corneal cell and/or skin cell culturing processes. They therefore provide a novel, efficient, cost effective and ethical means for corneal cell and/or skin cell production e.g. for tissue engineering or the growth of cellular structures.

MMC agents are a special class of food-grade, non-toxic, non-addictive, inert substances. They are typically generated as by-products in common agricultural, marine, fermentation and bio-fuel production processes, making them inexpensive and ideal supplements for cheaper cell culture media for high-yield tissue engineering/cell manufacture. MMC agents are also chemically defined, therefore their use as a serum replacement in cell culture media reduces the variability currently observed for processes reliant on serum. This provides a significant advantage for tissue engineering or the use of cells as industrial tools or products. Use of MMC agents in cell culture medium represents a new animal/xenobiotic-free method for increasing the efficiency of tissue engineering/cell manufacture, a strategy that can reduce (or even eliminate) the need for serum supplementation, making the product truly animal-free. The invention therefore addresses several challenges in this industry.

In one aspect, the invention therefore provides a serum-free or reduced-serum cell culture medium for in vitro cell culture, wherein the cell culture medium comprises a basal medium and one or more macromolecular crowding agents selected from the group consisting of: PEG8, carrageenan, PVP40, PVP360 and PEG35; or a combination thereof.

Suitably, the macromolecular crowding agent may be a combination of: PVP40 and PVP360; or PEG8 and PEG35.

The use of a serum-free or reduced-serum cell culture medium comprising a basal medium and one or more macromolecular crowding agents for in vitro cell culture is also provided, wherein:
a) the cell is a corneal cell and the macromolecular crowding agent is selected from the group consisting of: PEG8, Carrageenan, PVP40, PVP360, PEG35, Ficoll® 70 and Ficoll® 400; or a combination thereof; or
b) the cell is a skin cell and the macromolecular crowding agent is selected from the group consisting of: PEG8, PEG35, PVP40, PVP360, Ficoll® 70 and Ficoll® 400; or a combination thereof.

An in vitro serum-free or reduced-serum cell culture method is also provided, comprising culturing cells in a serum-free or reduced-serum cell culture medium comprising a basal medium and one or more macromolecular crowding agents, wherein:
a) the cells are corneal cells and the macromolecular crowding agent is selected from the group consisting of: PEG8, Carrageenan, PVP40, PVP360, PEG35, Ficoll® 70 and Ficoll® 400; or a combination thereof;
b) the cells are skin cells and the macromolecular crowding agent is selected from the group consisting of: PEG8, PEG35, PVP40, PVP360, Ficoll® 70 and Ficoll® 400; or a combination thereof.

Suitably, the cells may be corneal cells and the macromolecular crowding agent may be a combination of: PEG8 and PEG35; Ficoll® 70 and Ficoll® 400; or PVP40 and PVP360.

Suitably, the cells may be skin cells and the macromolecular crowding agent may be a combination of: PEG8 and PEG35; Ficoll® 70 and Ficoll® 400; or PVP40 and PVP360.

Suitably, the basal medium may be DMEM/F12.

Suitably, the cell culture medium may be a serum-free cell culture medium, optionally wherein the cell culture medium does not contain materials obtained from an animal.

Suitably, the serum-free cell culture medium may be a chemically defined cell culture medium.

Suitably, the cell culture medium may further comprise glutamine, optionally wherein the cell culture medium further comprises ascorbic acid, insulin, transferrin, selenium, and ethanolamine.

Suitably, the cell culture medium may comprise more than about 1 mM, but less than about 10 mM L-alanyl-L-glutamine dipeptide; and optionally:
a) more than about 0.1 mM, but less than about 10 mM ascorbic acid; and
b) more than about 1 mg/L, but less than about 100 mg/L insulin; and
c) more than about 0.5 mg/L, but less than about 10 mg/L transferrin; and
d) more than about 0.5 μg/L, but less than about 10 μg/L selenium; and
e) more than about 0.2 mg/L, but less than about 20 mg/L ethanolamine.

Suitably, the cell culture medium may further comprise penicillin and streptomycin.

A cell culture medium supplement for in vitro serum-free or reduced-serum cell culture is also provided, comprising one or more macromolecular crowding agents selected from the group consisting of: PVP40, carrageenan, PEG8, PVP360 and PEG35, Ficoll® 70 and Ficoll® 400; or a combination thereof, wherein the supplement further comprises: insulin, transferrin, selenium, ethanolamine, ascorbic acid and/or glutamine.

Suitably, the supplement may comprise:
a) insulin at a concentration that when the supplement is added to a basal medium the insulin is at a final concentration of more than about 1 mg/L, but less than about 100 mg/L in the resultant cell culture medium;
b) transferrin at a concentration that when the supplement is added to a basal medium the transferrin is at a final concentration of more than about 0.5 mg/L, but less than about 10 mg/L in the resultant cell culture medium;
c) selenium at a concentration that when the supplement is added to a basal medium the selenium is at a final concentration of more than about 0.5 μg/L, but less than about 10 μg/L in the resultant cell culture medium;
d) ethanolamine at a concentration that when the supplement is added to a basal medium the ethanolamine is at a final concentration of more than about 0.2 mg/L, but less than about 20 mg/L in the resultant cell culture medium;
e) ascorbic acid at a concentration that when the supplement is added to a basal medium the ascorbic acid is at a final concentration of more than about 0.1 mM, but less than about 10 mM in the resultant cell culture medium;
f) L-alanyl-L-glutamine dipeptide at a concentration that when the supplement is added to a basal medium the amount of glutamine available in the medium is at a final concentration of more than about 1 mM, but less than about 10 mM in the resultant cell culture medium; and
g) a macromolecular crowding agent selected from:
   (i) PEG8 at a concentration that when the supplement is added to a basal medium the PEG8 is at a final concentration of more than about 0.25 g/L, but less than about 25 g/L in the resultant cell culture medium; or
   (ii) PEG35 at a concentration that when the supplement is added to a basal medium the PEG35 is at a final concentration of more than about 0.5 g/L, but less than about 50 g/L in the resultant cell culture medium; or
   (iii) PVP40 at a concentration that when the supplement is added to a basal medium the PVP40 is at a final concentration of more than about 0.05 g/L, but less than about 50 g/L in the resultant cell culture medium; or
   (iv) PVP360 at a concentration that when the supplement is added to a basal medium the PVP360 is at a final concentration of more than about 50 mg/L, but less than about 15 g/L in the resultant cell culture medium.
   (v) Carrageenan at a concentration that when the supplement is added to a basal medium the Carrageenan is at a final concentration of more than about 1 mg/L, but less than about 10 g/L in the resultant cell culture medium; or
   (vi) Ficoll® 70 at a concentration that when the supplement is added to a basal medium the Ficoll® 70 is at a final concentration of more than about 300 mg/L, but less than about 300 g/L in the resultant cell culture medium; or
   (vii) Ficoll® 400 at a concentration that when the supplement is added to a basal medium the Ficoll® 400 is at a final concentration of more than about 300 mg/L, but less than about 300 g/L in the resultant cell culture medium.

Suitably, the supplement may comprise:
a) insulin at a concentration that when the supplement is added to a basal medium the insulin is at a final concentration of about 10 mg/L;
b) transferrin at a concentration that when the supplement is added to a basal medium the transferrin is at a final concentration of about 5.5 mg/L;
c) selenium at a concentration that when the supplement is added to a basal medium the selenium is at a final concentration of about 6.7 μg/L;
d) ethanolamine at a concentration that when the supplement is added to a basal medium the ethanolamine is at a final concentration of about 2 mg/L;
e) ascorbic acid at a concentration that when the supplement is added to a basal medium the ascorbic acid is at a final concentration of about 1 mM;
f) L-alanyl-L-glutamine dipeptide at a concentration that when the supplement is added to a basal medium the L-alanyl-L-glutamine dipeptide is at a final concentration of about 4 mM; and
g) a macromolecular crowding agent selected from the group consisting of: Carrageenan at a concentration that when the supplement is added to a basal medium the Carrageenan is at a final concentration of about 10 mg/L; PEG8 at a concentration that when the supplement is added to a basal medium the PEG8 is at a final concentration of about 1.1 g/L; PEG35 at a concentration that when the supplement is added to a basal medium the PEG35 is at a final concentration of about 2 g/L; PVP40 at a concentration that when the supplement is added to a basal medium the PVP40 is at a final concentration of about 4.5 g/L; PVP360 at a concentration that when the supplement is added to a basal medium the PVP360 is at a final concentration of about 10 g/L; and Ficoll® 70 and Ficoll® 400 at a concentration that when the supplements are added to a basal medium the Ficoll® 70 and Ficoll® 400 are at a final concentration of about 1 and 0.75 g/L, respectively.

Suitably, the supplement may be a liquid solution or a dry powder or a granulated dry powder.

Suitably, the supplement may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and a macromolecular crowding agent selected from the group consisting of: 0.5 g/L Carrageenan, 55 g/L PEG8, 225 g/L PVP40, 100 g/L PEG35, 500 g/L PVP360, and 50 and 37.5 g/L Ficoll® 70 and Ficoll® 400, respectively.

A hermetically-sealed vessel containing a serum-free or reduced-serum cell culture medium or a cell culture medium supplement described herein is also provided.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows human corneal stromal cell proliferation with MMC-supplemented media. Cell number was determined using the AlamarBlue™ viability assay after 7 days in culture. Bars represent averages of three independent repeats and error bars show SD. Statistical analysis was performed by two-way ANOVA and subsequent Bonferroni's multiple comparisons test with p-values of <0.05, 0.01, and 0.001 represented by *, **, and ***, respectively.

FIG. 2 shows human corneal stromal cell proliferation with MMC-supplemented media. Cell number was determined using the AlamarBlue™ viability assay at 2, 4, and 6 weeks of culture. Bars represent averages of three independent repeats and error bars show SD. Statistical analysis was performed by two-way ANOVA and subsequent Bonferroni's multiple comparisons test (Graphpad Prism) with p-values of <0.05 represented by *.

FIG. 3 shows the physical properties of tissues bio-fabricated for 6 weeks with Control, +PEG, and +PEG+PA media. A) The tissues' wet (left) and dry weight (right panel). B) Tissue hydration. C) Tissue thickness. Statistical analysis indicated that +PEG and +PEG+PA conditions promoted bio-fabrication, with supplements significantly increasing tissue density and thickness while reducing total hydration. Average of three independent repeats and corresponding SD are represented. Statistical analysis was performed by one-way ANOVA and subsequent Bonferroni's multiple comparisons test (Graphpad Prism) with p-values of <0.05 and 0.01 represented by * and **, respectively.

FIG. 4 shows analysis of tissue composition. Medium supplements +PEG and +PEG+PA promoted deposition of cornea-specific matrix collagens (type I and V) and proteoglycans. Average of three independent repeats and corresponding SD are represented. Statistical analysis was performed by one-way ANOVA and subsequent Bonferroni's multiple comparisons test (Graphpad Prism) with p-values of <0.05, 0.01 and 0.001 represented by *, ** and ***, respectively.

FIG. 5 shows skin fibroblasts were grown in DMEM/F12 media (SFM), serum-supplemented media (+1% FBS), or MMC-supplemented media for 3 days. Bars represent averages of three independent repeats and error bars show SD. Statistical analysis was performed utilising two-way ANOVA and subsequent Dunnet's multiple comparisons test with p<0.05, 0.01, and 0.001 represented by *, ** and *** respectively.

FIG. 6 shows skin fibroblasts were grown in DMEM/F12 media (SFM), serum-supplemented media (+1% FBS), or MMC-supplemented media for 7 days. Bars represent averages of three independent repeats and error bars show SD. Statistical analysis was performed utilising two-way ANOVA and subsequent Dunnet's multiple comparisons test with p<0.05, 0.01, and 0.001 represented by *, ** and *** respectively.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

In eukaryotic cell culture, MMC agents have previously been combined with high levels of serum supplementation (e.g., 2-20% v/v) to enhance and accelerate extracellular matrix (ECM) deposition (see for example references [1] and [10]). Their effect on cell culture depends on the specific MMC agent that is used. This may be due to the specific properties of each MMC agent (e.g., charge, size, hydrodynamic radius, etc.—see reference [8]).

The inventors have now investigated the effects of different MMC agents on cell culture in the absence of serum, or in reduced-serum conditions (e.g., 0.1-2%). They have surprisingly found that when specific MMC agents are used as cell culture supplements in the absence of serum, cell proliferation is increased, and/or cell differentiation is reduced, and/or tissue production is promoted. These MMCs can therefore advantageously be used in serum-free or reduced-serum culture conditions to improve cell culture processes. These effects appear to be dependent on the specific MMCs used as well as the cell type (see reference [7], which shows the deleterious effect of adding a Ficoll® 70/Ficoll® 400 mix to serum-free media during the culture of adipose stem cells).

A serum-free or reduced-serum cell culture medium for in vitro cell culture is therefore provided herein, wherein the cell culture medium comprises a basal medium and one or more macromolecular crowding agents selected from the group consisting of: PEG8, PEG35, PVP40, PVP360, Carrageenan, Ficoll® 70, and Ficoll® 400; or a combination thereof.

The term “cell culture” as used herein refers to keeping the cells in an artificial environment under conditions favouring growth, differentiation, and/or continued viability of the cells. Cell growth may be promoted for example if cell number and/or cell viability is increased as compared to a suitable control. Cell culture is assessed by number of viable cells/ml culture medium. The terms “cell culture” and “in vitro cell culture” are used interchangeably herein.

As would be known by a person of skill in the art, cells may be cultured for different time periods. In the context of the invention, cells may be cultured in serum-free or reduced-serum cell culture medium comprising a basal medium and one or more macromolecular crowding agents for at least one day. For example, the cells may be cultured in serum-free or reduced-serum cell culture medium comprising a basal medium and one or more macromolecular crowding agents for at least 2 days.

In one example, the cells may be cultured in serum-free or reduced-serum cell culture medium comprising a basal medium and one or more macromolecular crowding agents for at least 3 days, or at least 4 days. In another example, the cells may be cultured in serum-free or reduced-serum cell culture medium comprising a basal medium and one or more macromolecular crowding agents for at least 5 days, or at least 6 days.

In one example, the cells may be cultured in serum-free or reduced-serum cell culture medium comprising a basal medium and one or more macromolecular crowding agents for at least 7 days (i.e. at least a week). In another example, the cells may be cultured in serum-free or reduced-serum cell culture medium comprising a basal medium and one or more macromolecular crowding agents for at least 2 weeks, or at least 3 weeks. In another example, the cells may be cultured in serum-free or reduced-serum cell culture medium comprising a basal medium and one or more macromolecular crowding agents for at least 4 weeks, or at least 5 weeks. In another example, the cells may be cultured in serum-free or reduced-serum cell culture medium comprising a basal medium and one or more macromolecular crowding agents for at least 6 weeks.

Cell culture may be performed in any means known in the art. For example, the cells may be cultured in a cell culture vessel, such as a cell culture flask or a cell culture plate. Alternatively, cells may be cultured in a bioreactor.

The cells may be adherent cells, or they may be in suspension. For adherent cells, the cells may be attached to any suitable surface. For example, the cells may be attached to a surface of a cell culture plate or to a surface of a cell culture flask. Alternatively, the cells may be attached to microcarriers, such as gelatin, dextran, cellulose, plastic, or glass beads.

The terms “cell culture medium” and “culture medium” (plural “media” in each case) refer to a nutritive solution for cultivating live cells and may be used interchangeably. Typically, the cell culture medium may be a complete formulation, i.e., a cell culture medium that requires no further supplementation to culture cells. Various cell culture media will be known to those skilled in the art, who will also appreciate that the type of cells to be cultured may dictate the type of culture medium to be used.

Characteristics and formulations of cell culture media vary depending upon the particular cellular requirements. Important parameters include osmolarity, pH, and nutrient compositions. Cell culture medium formulations have been well documented in the literature and a large number of media are commercially available. In early cell culture work, medium formulations were based upon the chemical composition and physicochemical properties (e.g., osmolality, pH, etc.) of blood and were referred to as “physiological solutions”. However, cells in different tissues of a mammalian body are exposed to different microenvironments with respect to oxygen/carbon dioxide partial pressure and concentrations of nutrients, vitamins, and trace elements; accordingly, successful in vitro culture of different cell types may require the use of different medium formulations. Typical components of cell culture media include amino acids, organic and inorganic salts, vitamins, trace metals, sugars, lipids and nucleic acids, the types and amounts of which may vary depending upon the particular requirements of a given cell or tissue type. Cell culture media is made up of compatible components that are maintained together in solution and form a “stable” combination. A solution containing “compatible ingredients” or “compatible components” is said to be “stable” when the ingredients do not precipitate, degrade or decompose substantially over the standard shelf-life of the solution.

A cell culture medium as described herein typically comprises a basal medium and one or more macromolecular crowding (MMC) agents. A “basal medium” (plural “media”) is a cell culture reagent that is used as the initial (starting) medium to which supplements (such as growth factors etc) are added to generate a cell culture media that is suitable for supporting cell growth, without further supplementation. Basal medium is a medium that is typically useful only for cell nutrition, but not for the maintenance of cell viability, growth or production of product. It typically comprises a number of ingredients, including amino acids, sugars, lipids, vitamins, organic and inorganic salts, and buffers, each ingredient being present in an amount which supports the maintenance of a mammalian cell in vitro. Merely by way of example and not limitation, examples of basal media include: Dulbecco's Modified Eagle's Medium (DMEM), Ham's F-12 (F-12), Minimal Essential Medium (MEM), Basal Medium Eagle (BME), RPMI-1640, Ham's F-10, αMinimal Essential Medium (αMEM), Glasgow's Minimal Essential Medium (G-MEM), and Iscove's Modified Dulbecco's Medium (IMDM), or any combination thereof.

In a particular example, the basal medium may be DMEM or F-12, or a combination thereof (e.g. DMEM/F12).

Other media that are commercially available (e.g., from Invitrogen Corporation, Carlsbad, CA) or that are otherwise known in the art can be equivalently used as a basal medium including, but not limited to, 293 SFM, CD-CHO medium, VP SFM, BGJb medium, Brinster's BMOC-3 medium, cell culture freezing medium, CMRL media, EHAA medium, eRDF medium, Fischer's medium, Gamborg's B-5 medium, GLUTAMAX™ supplemented media, Grace's insect cell media, HEPES buffered media, Richter's modified MEM, IPL-41 insect cell medium, Leibovitz's L-15 media, McCoy's 5A media, MCDB 131 medium, Media 199, Modified Eagle's Medium (MEM), Medium NCTC-109, Schneider's Drosophila medium, TC-100 insect medium, Waymouth's MB 752/1 media, William's Media E, protein free hybridoma medium II (PFHM II), AIM V media, Keratinocyte SFM, defined Keratinocyte SFM, STEMPRO® SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX bone marrow karyotyping medium, KNOCKOUT D-MEM and CO2 independent medium. The above media are obtained from manufacturers known to those of ordinary skill in the art, such as JRH, Sigma, HyClone, and BioWhittaker.

Serum is commonly added as a supplement to cell culture media to support the growth of cells in culture. The term “serum” as used herein refers to the serum component of blood i.e. the plasma from which the clotting proteins have been removed. It also encompasses “reconstituted” serum (e.g. serum that has been further treated to remove undesirable (e.g. deleterious) components and optionally concentrate beneficial components).

Typically, the serum is from bovine origin (fetal bovine serum, FBS; bovine calf serum, BCS), caprine origin (goat serum, GS), or equine origin (horse serum, HS). While FBS is the most commonly applied supplement in animal cell culture media, other serum sources are also routinely used, including newborn calf, horse and human. These types of chemically undefined supplements serve several useful functions in cell culture media. For example, serum provides additional nutrients (both in the solution as well as bound to the proteins) for cells. It also provides several growth factors and hormones involved in growth promotion and specialized cell function. Furthermore, it provides several binding proteins like albumin, transferrin, which can carry other molecules into the cell. For example: albumin carries lipids, vitamins, hormones, etc. into cells. It also supplies proteins, like fibronectin, which promote the attachment of cells to the substrate. It also provides spreading factors that help the cells to spread out before they begin to divide. Serum also provides protease inhibitors which protect cells from proteolysis. It also provides minerals, like Na+, K+, Zn2+, Fe2+, etc. It increases the viscosity of the medium and thus, protects cells from mechanical damage during agitation of suspension cultures. It also acts a buffer.

Although there are several advantages to using serum as a supplement during cell culture, there are also several occasions when it may be desired to reduce or omit serum from the cell culture medium during cell culture. For example, the presence of serum in cell culture media introduces variability in the composition of the media, as serum is by its very nature variable in its composition. Testing also needs to be performed to maintain the quality of each batch of serum before it is used. In addition, serum may contain some growth inhibiting factors and its presence in cell culture media may interfere with the differentiation of adult stem or progenitor cells, as well as with the purification and isolation of cell culture products. Finally, it is not from a sustainable source and thus is a relatively expensive supplement for cell culture on a commercial scale.

Serum supplements can also be contaminated with infectious agents (e.g., mycoplasma and viruses) which can seriously undermine the health of the cultured cells and the quality of the final product. The use of undefined components such as serum or animal extracts also prevents the true definition and elucidation of the nutritional and hormonal requirements of the cultured cells, thus eliminating the ability to study, in a controlled way, the effect of specific growth factors or nutrients on cell growth and differentiation in culture. Moreover, serum supplementation of culture media can complicate and increase the costs of the purification of the desired substances from the culture media due to nonspecific co-purification of serum or extract proteins.

Variability in the composition of serum, risk of contamination, and its lack of sustainable sources are issues for tissue engineering and industrial cell manufacture on a commercial and global scale.

Serum-free methods of cell culture are available, but typically result in lower levels of cell proliferation and/or cell maintenance, or require the addition of cocktails of recombinant proteins, growth factors and other costly ingredients specific to each cell type.

The invention is based on the surprising finding that supplementing cell culture media with MMCs such as one or more MMC agent selected from the group consisting of: PEG8, PEG35, PVP40, PVP360, Carrageenan, Ficoll® 70, and Ficoll® 400; or a combination thereof, can, for certain cells, mitigate the need for serum in the cell media.

The cell culture medium described herein is therefore typically a serum-free cell culture medium or a reduced-serum cell culture medium.

The term “reduced-serum” cell culture medium is used to describe a cell culture medium for use in cell culture, wherein the cell culture medium has been supplemented with serum, but at a lower level than would usually be used for optimal culture of the cells of interest. For example, it is accepted in the field that corneal cells or skin cells are typically cultured in a cell culture medium that comprises at least 5% (v/v) serum for cell growth, and tissue formation. In the context of cell culture of corneal cells or skin cells, a culture medium having no more than about 2% (v/v) serum may therefore be considered a “reduced-serum” cell culture medium. In other words, a reduced-serum cell culture medium for use in culturing corneal cells or skin cells may have a maximum serum concentration of about 2% (v/v) serum. For example, in the context of corneal cells or skin cells, a reduced-serum cell culture medium may have a serum concentration that is in the range of from (substantially) no serum to no more than about 2% (v/v) serum. In other words, the reduced-serum cell culture medium described herein may have a serum concentration that is in the range of from (substantially) no serum to a maximum of about 2% (v/v) serum.

In some examples, a reduced-serum cell culture medium may have a serum concentration that is in the range of from (substantially) no serum to a maximum of about 1.5% (v/v) serum.

In another example, the reduced-serum cell culture medium may have a serum concentration that is in the range of from (substantially) no serum to a maximum of about 1% (v/v) serum. In a further example, the reduced-serum cell culture medium may have a serum concentration that is in the range of from (substantially) no serum to a maximum of about 0.5% (v/v) serum.

A reduced-serum cell culture medium may have a maximum serum concentration of about 2% (v/v) serum. Alternatively, it may have a maximum serum concentration of about 1.5% (v/v) serum.

For example, it may have a maximum serum concentration of about 1% (v/v) serum. As a further example, it may have a maximum serum concentration of about 0.5% (v/v) serum.

The reduced-serum cell culture medium may have (substantially) no serum. In this context, “substantially no serum” means that the cell culture medium may have no more than trace amounts of serum. Trace amounts may be defined as a maximum of 0.1% (v/v) serum in the cell culture medium.

For example, the reduced-serum cell culture medium may have zero to about 2% (v/v) serum. For example, the reduced-serum cell culture medium may have zero to about 1.5% (v/v) serum. For example, the reduced-serum cell culture medium may have zero to about 1% (v/v) serum. For example, the reduced-serum cell culture medium may have zero to about 0.5% (v/v) serum. For example, the reduced-serum cell culture medium may have about 0.1% to about 2% (v/v) serum. For example, the reduced-serum cell culture medium may have about 0.1% to about 1.5% (v/v) serum. For example, the reduced-serum cell culture medium may have about 0.1% to about 1% (v/v) serum. As another example, the reduced-serum cell culture medium may have about 0.1% to about 0.5% (v/v) serum.

Cell culture media wherein there is no detectable serum are referred to herein as “serum-free” cell culture media (SFM). Typically, for media that contains serum, the serum is added as a supplement at the start of, or during cell culture. The term “serum-free” cell culture medium therefore includes cell culture media which have not been supplemented with serum. The term “serum-free” is very well known in the art.

As would be clear to a person of skill in the art, “serum-free” media may comprise a number of additives and supplements, provided that it does not contain detectable levels of serum. Two different serum-free media are tested in the examples section below, both of which are encompassed by the term “serum-free” media; “SFM” (where “SFM” is used to describe media comprising basal media (e.g. DMEM/F12) with glutamine (e.g. GlutaMAX) as the main supplement (with antibiotics)); and “SFM*” (where “SFM*” is used to describe media comprising basal media (e.g. DMEM/F12) with glutamine (e.g. GlutaMAX), ascorbic acid, insulin, transferrin, selenium and ethanolamine as the main supplements (with antibiotics)).

SFM and SFM* are both examples of serum-free media that can be used in the context of the invention.

The serum-free or reduced-serum cell culture media described herein are particularly advantageous as they provide a more chemically defined media for cell culture, using reagents that are more sustainable, and with a lower risk of contamination compared to equivalents that are reliant on serum.

Serum-free media can still contain one or more of a variety of animal-derived components, including albumin, fetuin, various hormones and other proteins.

In one example, the serum-free cell culture medium does not contain materials obtained from an animal. In other words, in this example, the medium does not contain animal derived material. The term “animal derived” material as used herein refers to material that is derived in whole or in part from an animal source, including recombinant animal DNA or recombinant animal protein. In other words, the material is obtained from, or isolated from, an animal source. For the avoidance of doubt, synthetic materials (e.g. proteins) that mimic materials (e.g. proteins) that are naturally found in animals are not “animal derived” as they have been synthetically made and are chemically defined.

For example, the serum-free cell culture medium may be a chemically defined cell culture medium. A “chemically defined” medium is one in which each chemical species and its respective quantity is known prior to its use in culturing cells. A chemically defined medium is made without lysates or hydrolysates whose chemical species are not known and/or quantified.

Chemically defined media are often specifically formulated to support the culture of a single cell type, contain no undefined supplements and instead incorporate defined quantities of purified growth factors, proteins, lipoproteins and other substances usually provided by serum. Since the components (and concentrations thereof) in such culture media are precisely known, these media are generally referred to as “defined culture media.” The distinction between serum-free media and defined media is that serum-free media is devoid of serum and protein fractions (e.g., serum albumin), but not necessarily of other undefined components such as organ/gland extracts. Serum-free media thus cannot be considered to be defined media in the true definition of the term.

Defined media generally provide several distinct advantages to the user. For example, the use of defined media facilitates the investigation of the effects of a specific growth factor or other medium component on cellular physiology, which can be masked when the cells are cultivated in serum- or extract-containing media. In addition, defined media typically contain much lower quantities of protein (indeed, defined media are often termed “low protein media”) than those containing serum or extracts, rendering purification of biological substances produced by cells cultured in defined media far simpler and less expensive.

Most defined media incorporate into the basal media additional components to make the media more nutritionally complex, whilst maintaining the serum-free and low protein content of the media. Examples of such components include bovine serum albumin (BSA) or human serum albumin (HSA); certain growth factors derived from natural (animal) or recombinant sources such as epidermal growth factor (EGF) or fibroblast growth factor (FGF); lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements.

The cell culture media described herein is a serum-free cell culture medium or a reduced-serum cell culture medium, optionally wherein the serum-free cell culture medium does not contain materials obtained from an animal (e.g. is a chemically-defined cell culture medium).

The cell culture media described herein may include one or more additional components. Examples of such components include synthetic components, such as but not limited to one or more of: synthetic serum albumin, Lonza HL-1™ supplement, synthetic fibroblast growth factor (FGF), synthetic epidermal growth factor (EGF), synthetic platelet-derived growth factor (PDGF), human growth factor (HGF), transforming growth factor (TGF), insulin-like growth factor (IGF), keratinocyte growth factor (KGF), insulin, transferrin, N-2 MAX media and N21-MAX media supplement (R&D), B-27™ supplement (ThermoScientific), hybridoma supplement (Grisp), panexin basic, panexin CD, panexin NTA, panexin NTS, panexin BMM (Pan Biotech), alpha-1-anti-trypsin, alpha-1-acid glycoprotein, alpha-2-macroglobulin, beta-2-microglobulin, haptoglobin, plasminogen, carbonic anhydrase I, carbonic anhydrase II, ferritin, C-reactive protein, fibrinogen, hemoglobin A, hemoglobin beta A2, hemoglobin beta C, hemoglobin beta F, hemoglobin beta S, thyroglobulin, bilirubin, creatinin, cortisol, growth hormone, parathormone, triiodothyronine, thyroxine (T4), thyroid-stimulating hormone (TSH), follicle-stimulating hormone (FSH), testosterone, progesterone (P4), prolactin, luteinizing hormone, prostaglandin E, prostaglandin F, cholesterol, lactate-dehydrogenase, and/or alkaline phosphatase.

As stated elsewhere herein, the cell culture medium typically comprises a basal medium and one or more macromolecular crowding (MMC) agents. In particular the cell culture media described herein comprise one or more macromolecular crowding agents selected from the group consisting of: PEG8, PEG35, PVP40, PVP360, Carrageenan, Ficoll® 70, and Ficoll® 400; or a combination thereof.

Macromolecular crowding (MMC) is a biophysical phenomenon based on the principles of excluded-volume effect. It involves the addition of macromolecules to reaction or culture media. Following the principles of excluded volume effect (two molecules cannot occupy the same space at the same time), MMC significantly increases rates and kinetics of biochemical reactions and biological processes. According to the excluded volume effect theory, the volume of a solution that is excluded to a particular molecule is dependent on the sum of nonspecific hindrances (governed by size and shape) and electrostatic repulsions (governed by electrical charge) between the background molecules. Crowding is a result of the reduction of the available solvent volume by a macromolecule, which can be mobile or fixed. Crowding hinders solute diffusion, thereby increasing the effective solute concentration. This, in turn, increases the chemical potential of the solute. Crowding can therefore shift reaction equilibria and change the rates of chemical reactions. Crowding has therefore been used extensively to study polymer looping dynamic properties, DNA structure, condensation, replication, and stability, for example. Macromolecular crowding influences many critical processes including cell adhesion, migration, proliferation as well as extracellular matrix formation and remodelling [7]. These effects have been shown herein to positively affect cell culture of fat and muscle cells (or combinations thereof). Use of such MMC agents during cell culture of fat and/or muscle cells as described herein is therefore particularly advantageous.

Several MMC agents are known. In the context of the cell culture media, supplements, methods and uses described herein, the MMC agent may be one or more MMC agents selected from the group consisting of: PEG8, PEG35, PVP40, PVP360, Carrageenan, Ficoll® 70, and Ficoll® 400; or a combination thereof.

Polyethylene glycol (PEG) is a commonly used macromolecular crowder having effects on in vitro experiments, such as influencing ligand affinity and rate of enzymatic reaction and promoting extracellular matrix deposition when used with serum [8]. Polyethylene glycol is prepared by polymerization of ethylene oxide and is commercially available over a wide range of molecular weights, from 300 Da to 10,000 kDa (Alister et al., Angewandte Chemie International Edition Volume 48, Issue 7 p. 1248-1252). For example, Polyethylene Glycol 8 kDa (PEG8) is a non-toxic polyether with hydrophilic head allowing to dilution in aqueous solutions [4]. Macromolecular crowding induced by PEG8 can modulate reactions by increasing substrate binding at high concentrations as well as increasing proliferation bacterial strains [5, 6]. The molecular structure of PEG8 is well known, see for example Sigma-Aldrich, Cas Number 25322-68-3, linear formula: H(OCH₂CH₂)ₙOH and Hyun-Jun Jang et al., Toxicol Res. 2015 June; 31(2): 105-136.

An alternative PEG that may be used herein is PEG35. The molecular structure of PEG35 is also well known, see for example Hyun-Jun Jang et al., Toxicol Res. 2015 June; 31(2): 105-136.

Polyvinylpyrrolidone 40 kDa (PVP40) is a water soluble polymer with a variety of uses including in beverage stabilization and medical uses where it is used as plasma volume expander [9]. PVP40 in combination with serum has also been shown to be an effective macromolecular crowder with treatment increasing both collagen type I and proliferation of human dermal fibroblasts [10]. The molecular structure of PVP40 is well known, see for example Sigma Aldrich, Cas Number 9003-39-8, linear formula (C₆H₉NO)ₙ and Kariduraganavar et al., Natural and Synthetic Biomedical polymers; chapter 1, 2014, pages 1 to 31.

Polyvinylpyrrolidone 360 kDa (PVP360) used as a macromolecular crowder together with serum has been shown to increase human dermal fibroblast cell proliferation and extracellular matrix deposition as indicated by increases in collagen type I production [10]. The molecular structure of PVP360 is also well known. See for example Kariduraganavar et al., Natural and Synthetic Biomedical polymers; chapter 1, 2014, pages 1 to 31.

Carrageenans (also known as carrageenans) are a family of natural linear sulphated polysaccharides that are extracted from red edible seaweeds. The most well-known and still most important red seaweed used for manufacturing the hydrophilic colloids to produce carrageenan is Chondrus crispus (Irish moss) which is a dark red parsley-like plant that grows attached to the rocks. Carrageenans are widely used in the food industry, for their gelling, thickening, and stabilizing properties. Their main application is in dairy and meat products, due to their strong binding to food proteins.

All carrageenans are high-molecular-weight polysaccharides and mainly made up of alternating 3-linked b-D-galac-topyranose (G-units) and 4-linked a-D-galactopyranose (D-units) or 4-linked 3,6-anhydro-a-D-galactopyranose (DA-units), forming the disaccharide repeating unit of carrageenans. There are three main commercial classes of carrageenan: Kappa carrageenan, Iota carrageenan and Lambda carrageenan. The molecular structures of different types of carrageenan are well known, see for example Hilliou, Adv Food Nutr Res. 2014; 72:17-43.

In a preferred example, the carrageenan used in the context of the invention is lambda carrageenan. Carrageenans encompass a family of sulphated galactans originally extracted from red seaweed, where they have been found to play key structural functions. Traditionally, carrageenans are produced and used as crude extracts comprising different combinations of three molecular species defined by their sulphation and the presence or absence of anhygalactose. The lambda-carrageenan contains about of 35% ester sulfate and no anhygalactose, making it highly soluble in water and unable to form gels. In contrast, the iota-carrageenan and kappa-carrageenan contain less ester sulfate and about 30-35% of 3,6-anhydrogalactose, making them insoluble in cold water and forming thermo-reversible gels in hot aqueous solutions. The different physicochemical and biological properties of lambda-carrageenan demonstrates that this molecular species is altogether distinct from the iota and kappa species, as well as from crude carrageenan extracts.

Carrageenan has been proposed to be a promising macromolecular crowder (MMC) for tissue engineering due to its ability to increase extracellular matrix production. Treatment of adipose-derived stem cells with carrageenan and serum has been shown to enhance extracellular matrix deposition of collagen type I, II and V, to increase cell proliferation as well as increasing osteogenesis, chondrogenesis and decreasing adipogenesis [1].

Ficoll® 70 (also known as Poly(sucrose-co-epichlorhydrin)) is used as a macromolecular crowding agent in studies of cell volume signaling and protein refolding. It may be used in tissue engineering and macromolecular conformation research for the development, evaluation and use of macromolecular crowding (MMC) systems and configurations. The molecular structure of Ficoll® 70 is well known, see for example Sigma Aldrich, Cas Number 72146-89-5, and CN102690364A.

Ficoll® 400 (also known as Polysucrose 400) is a non-ionic synthetic polymer of sucrose used for cell separation and organ isolation. The molecular structure of Ficoll® 400 is well known, see for example Sigma Aldrich, Cas Number 26873-85-8 and CN102690364A and https://pubchem.ncbi.nlm.nih.gov/compound/Ficoll-400.

The MMC agents described herein may be used as a single supplement (wherein only one MMC agent is added to the cell culture medium) or they may be used in combination. Suitable combinations may be identified by a person of skill in the art. For example, a combination of at least two MMC agents may be used. Alternatively, a combination of at least three or at least four MMC agents may be used.

Specific combinations of MMC agents are described herein, for example a combination of PVP40 and PVP360, or a combination of PEG8 and PEG35, or a combination of Ficoll® 70 and Ficoll® 400. The utility of these specific combinations is demonstrated herein for specific cell types. However, other suitable combinations may also be selected by a person of skill in the art based on the disclosure herein. For example, PVP40 may be combined with PEG8. Alternatively, PVP40 may be combined with PEG35. Alternatively, PVP40 may be combined with Ficoll® 70. Alternatively, PVP40 may be combined with Ficoll® 400. Alternatively, PVP40 may be combine with Carrageenan.

In another example, PVP360 may be combined with PEG8. Alternatively, PVP360 may be combined with PEG35. Alternatively, PVP360 may be combined with Ficoll® 70. Alternatively, PVP360 may be combined with Ficoll® 400. Alternatively, PVP360 may be combine with Carrageenan.

In another example, PEG8 may be combined with Ficoll® 70. Alternatively, PEG8 may be combined with Ficoll® 400. Alternatively, PEG8 may be combine with Carrageenan.

In another example, PEG35 may be combined with Ficoll® 70. Alternatively, PEG35 may be combined with Ficoll® 400. Alternatively, PEG35 may be combine with Carrageenan.

The MMC agents may be used at any appropriate concentration within the cell culture media described herein.

For example, when PEG8 is used, it may be used at a final concentration of more than about 0.25 g/L, but less than about 25 g/L cell culture medium. For example, cell culture media for culturing corneal cells or skin cells may comprise a final concentration of PEG8 of more than about 0.5 g/L, but less than about 10 g/L. For example, it may typically comprise more than about 0.5 g/L, but less than about 5 g/L. Typically, cell culture media for culturing corneal cells or skin cells has a final concentration of PEG8 of about 1.1 g/L.

In another example, when PEG35 is used, it may be used at a final concentration of more than about 0.5 g/L, but less than about 50 g/L cell culture medium. For example, cell culture media for culturing corneal cells or skin cells may comprise a final concentration of PEG35 of more than about 0.5 g/L, but less than about 25 g/L. For example, it may typically comprise more than about 1 g/L, but less than about 10 g/L. Typically, cell culture media for culturing corneal cells or skin cells has a final concentration of PEG35 of about 2 g/L.

In a further example, when PVP40 is used, it may be used at a final concentration of more than about 0.05 g/L, but less than about 50 g/L cell culture medium. For example, cell culture media for culturing corneal cells or skin cells may comprise a final concentration of PVP40 of more than about 0.5 g/L, but less than about 25 g/L. For example, it may typically comprise more than about 1 g/L, but less than about 10 g/L. Typically, cell culture media for culturing corneal cells or skin cells has a final concentration of PVP40 of about 4.5 g/L.

In a further example, when PVP360 is used, it may be used at a final concentration of more than about 50 mg/L, but less than about 15 g/L cell culture medium. For example, cell culture media for culturing corneal cells or skin cells may comprise a final concentration of PVP360 of more than about 0.5 g/L, but less than about 15 g/L. For example, it may typically comprise more than about 1 g/L, but less than about 15 g/L. Typically, cell culture media for culturing corneal cells or skin cells has a final concentration of PVP360 of about 10 g/L.

For example, when carrageenan is used, it may be used at a final concentration of more than about 1 mg/L, but less than about 10 g/L of cell culture medium. For example, cell culture media for culturing corneal cells or skin cells may comprise a final concentration of carrageenan of more than about 1 mg/L, but less than about 10 g/L. For example, it may typically comprise more than about 1 mg/L, but less than about 1 g/L. For example, it may typically comprise more than about 5 mg/L, but less than about 100 mg/L. For example, it may typically comprise more than about 5 mg/L, but less than about 50 mg/L. Typically, cell culture media for culturing corneal cells or skin cells has a final concentration of carrageenan of about 10 mg/L.

For example, when Ficoll® 70, Ficoll® 400, or a combination thereof is used, it may be used at a final concentration of more than about 300 mg/L, but less than about 300 g/L of cell culture medium. For example, cell culture media for culturing corneal cells or skin cells may comprise a final concentration of Ficoll® 70, Ficoll® 400, or a combination thereof of more than about 300 mg/L, but less than about 300 g/L. For example, it may typically comprise more than about 500 mg/L, but less than about 100 g/L of Ficoll® 70. For example, it may typically comprise more than about 375 mg/L, but less than about 75 g/L of Ficoll® 400. For example, it may typically comprise more than about 500 mg/L and 375 mg/L, but less than about 100 g/L and 75 g/L of Ficoll® 70 and Ficoll® 400, respectively. Typically, cell culture media for culturing corneal cells or skin cells has a final concentration of about 10 g/L of Ficoll® 70 and 7.5 g/L of Ficoll® 400.

Suitable final concentrations of MMC agents that are used in combination may be determined based on the disclosure provided herein, using routine methods known in the art.

The cell culture media described herein are particularly beneficial for cell culture of corneal cells or skin cells. In other words, these cell culture media are particularly useful for corneal or skin tissue engineering or corneal or skin cell manufacture.

The term “cell” as used herein refers includes all types of eukaryotic cells. In preferred embodiments, the term refers to mammalian cells, and most preferably a mouse or a human. The cells can be normal cells or abnormal cells (i.e., transformed cells, established cells, or cells derived from diseased tissue samples). The term includes both adherent and non-adherent cells.

Corneal cells include corneal stromal cells. Examples of suitable corneal stromal cells include corneal fibroblasts. Corneal fibroblasts, also known as keratocytes, are mesenchymal-derived cells of the corneal stroma. The terms “corneal fibroblast” and “corneal keratocyte” are used interchangeably herein. The corneal stromal cells may also be corneal stem cells preferably comprising limbal epithelial cells, i.e. a heterogeneous mixture of stem cells and differentiated cells which is obtainable from the limbus at the edge of the cornea. In another example, the stromal cells include stromal progenitor cells such as corneal fibroblasts (keratocytes) in a differentiated or undifferentiated form. Preferably, these corneal fibroblasts are obtained from the peripheral limbus or from limbal rings. In another example, the stromal cells are bone marrow cells. The stromal cells may also be derived from iPSCs.

Skin cells include epithelial cells and dermal fibroblasts. Dermal fibroblasts are found within the dermis layer of skin, and are responsible for generating connective tissue which organizes the stratified squamous epithelial cells of the epidermis into a unified tissue.

As described in the examples section below, a serum-free or reduced-serum cell culture medium comprising a basal medium and one or more macromolecular crowding agents selected from the group consisting of: PEG8, PEG35, PVP40, PVP360, Carrageenan, Ficoll® 70, and Ficoll® 400; or a combination thereof is particularly useful when culturing corneal cells, such as corneal stromal cells.

A combination of these recited MMC agents may be used for the culture of corneal cells, such as corneal stromal cells. For example, a combination of PEG8 and PEG35 may be advantageous for the culture of corneal cells, such as corneal stromal cells. In another example, a combination of PEG8 and PVP40 may be of benefit. In one example, a combination of PEG8 and PVP360 may be used. Alternatively, a combination of PEG8 and Ficoll® 70 may be advantageous. In one example, a combination of PEG8 and Ficoll® 400 may be used.

Alternatively, a combination of PEG35 and PVP40 may be of benefit in the culture of corneal cells, such as corneal stromal cells. In one example, a combination of PEG35 and PVP360 may be used. Alternatively, a combination of PEG35 and Ficoll® 70 may be advantageous. In one example, a combination of PEG35 and Ficoll® 400 may be used.

Alternatively, a combination of PVP40 and PVP360 may be used for the culture of corneal cells, such as corneal stromal cells. Alternatively, a combination of PVP40 and Ficoll® 70 may be advantageous. In one example, a combination of PVP40 and Ficoll® 400 may be used.

Alternatively, a combination of Carrageenan and PEG8 may be used. Alternatively, a combination of Carrageenan and PEG35 may be advantageous. In another example, a combination of Carrageenan and PVP40 may be of benefit. In one example, a combination of Carrageenan and PVP360 may be used. In another example, a combination of Carrageenan and Ficoll® 70 may be advantageous. In one example, a combination of Carrageenan and Ficoll® 400 may be used.

Alternatively, a combination of Ficoll® 70 and Ficoll® 400 may be used for the culture of corneal cells, such as corneal stromal cells.

In another example, a serum-free or reduced-serum cell culture medium comprising a basal medium and one or more macromolecular crowding agents selected from the group consisting of: PEG8, PEG35, PVP40, PVP360, Ficoll® 70 and Ficoll® 400; or a combination thereof is particularly useful when culturing skin cells, such as fibroblast cells.

A combination of these recited MMC agents may be used for the culture of skin cells, such as fibroblast cells. For example, a combination of PVP360 and PEG8 may be used. Alternatively, a combination of PVP360 and PEG35 may be advantageous. In another example, a combination of PVP360 and PVP40 may be of benefit. Alternatively, a combination of PVP360 and Ficoll® 70 may be advantageous. In another example, a combination of PVP360 and Ficoll® 400 may be of benefit.

In a further example, a combination of PEG8 and PEG35 may be advantageous for the culture of skin cells, such as fibroblast cells. In another example, a PEG8 and Ficoll® 70 may be advantageous. In another example, a combination of PEG8 and Ficoll® 400 may be of benefit.

Alternatively, a combination of PEG35 and PVP40 may be of benefit in the culture of skin cells, such as fibroblast cells. In another example, a PEG35 and Ficoll® 70 may be advantageous. In another example, a combination of PEG35 and Ficoll® 400 may be of benefit.

Alternatively, a combination of Ficoll® 70 and Ficoll® 400 may be used for the culture of skin cells, such as fibroblast cells.

Suitable concentrations for the MMC agents and the MMC agent combinations referred to above may be identified by a person of skill in the art.

As would be clear to a person of skill in the art, the cell culture medium described herein may include additional supplements, other than the basal medium and MMC agent(s) recited above.

For example, the cell culture medium may comprise glutamine (also referred to as stable glutamine herein). Cell culture media comprising a basal media (e.g. DMEM/F12) and glutamine (and with no added serum) is referred to in the examples section below as “SFM”.

Suitable sources of glutamine are well known in the art and may be readily identified by a person of skill in the art. By way of example, but not by way of limitation, glutamine may be present within the cell culture medium by the addition of GlutaMAX™, L-alanyl-L-glutamine dipeptide, L-glutamine or any other source of glutamine to a basal medium, such as DMEM/F12.

For example, cell culture media for culturing corneal cells or skin cells may comprise more than about 1 mM, but less than about 10 mM glutamine (e.g. L-alanyl-L-glutamine dipeptide). For example, it may typically comprise more than about 1.5 mM, but less than about 5 mM glutamine (e.g. L-alanyl-L-glutamine dipeptide). Typically, cell culture media for culturing corneal cells or skin cells has between about 2 mM to about 4 mM glutamine.

The cell culture media may comprise glutamine as recited above. In addition, it may comprise one or more of ascorbic acid, insulin, transferrin, selenium, and ethanolamine. Cell culture media comprising a basal media (e.g. DMEM/F12), glutamine ascorbic acid, insulin, transferrin, selenium, and ethanolamine (and no serum) is referred to in the examples section below as “SFM*”.

For example, cell culture media for culturing corneal cells or skin cells may comprise more than about 0.1 mM, but less than about 10 mM ascorbic acid. For example, it may typically comprise more than about 0.5 mM, but less than about 5 mM ascorbic acid, or more than about 0.5 mM, but less than about 2 mM ascorbic acid. Typically, cell culture media for culturing corneal cells or skin cells has about 1 mM ascorbic acid.

The cell culture media may comprise glutamine as recited above, in addition to ascorbic acid.

The cell culture media may therefore comprise glutamine in the ranges cited herein, and ascorbic acid at the ranges cited herein. As one example, for culturing corneal cells or skin cells it may have between about 2 mM to about 4 mM glutamine and about 1 mM ascorbic acid.

Cell culture media for culturing corneal cells or skin cells may comprise more than about 1 mg/L, but less than about 100 mg/L insulin. For example, it may typically comprise more than about 5 mg/L, but less than about 50 mg/L insulin, or more than about 5 mg/L, but less than about 25 mg/L insulin. Typically, cell culture media for culturing corneal cells or skin cells has about 10 mg/L insulin.

The cell culture media may comprise glutamine and/or ascorbic acid as recited above, in addition to insulin.

The cell culture media may therefore comprise glutamine in the ranges cited herein, ascorbic acid at the ranges cited herein and insulin ranges cited herein. As one example, for culturing corneal cells or skin cells it may have between about 2 mM to about 4 mM glutamine, about 1 mM ascorbic acid and about 10 mg/L insulin.

Cell culture media for culturing corneal cells or skin cells may comprise more than about 0.5 mg/L, but less than about 10 mg/L transferrin. For example, it may typically comprise more than about 1 mg/L, but less than about 8 mg/L transferrin, or more than about 3 mg/L, but less than about 8 mg/L transferrin. Typically, cell culture media for culturing corneal cells or skin cells has about 5.5 mg/L transferrin.

The cell culture media may comprise glutamine, ascorbic acid and/or insulin as recited above, in addition to transferrin.

The cell culture media may therefore comprise glutamine in the ranges cited herein, ascorbic acid at the ranges cited herein, insulin ranges cited herein and transferrin ranges cited herein. As one example, for culturing corneal cells or skin cells it may have between about 2 mM to about 4 mM glutamine, about 1 mM ascorbic acid, about 10 mg/L insulin, and about 5.5 mg/L transferrin.

Cell culture media for culturing corneal cells or skin cells may comprise more than about 0.5 μg/L, but less than about 10 μg/L selenium. For example, it may typically comprise more than about 2 μg/L, but less than about 10 μg/L selenium, or more than about 2 μg/L, but less than about 8 μg/L selenium. Typically, cell culture media for culturing corneal cells or skin cells has about 6.7 μg/L selenium.

The cell culture media may comprise glutamine, ascorbic acid, insulin and/or transferrin as recited above, in addition to selenium.

The cell culture media may therefore comprise glutamine in the ranges cited herein, ascorbic acid at the ranges cited herein, insulin ranges cited herein, transferrin ranges cited herein and selenium ranges cited herein. As one example, for culturing corneal cells or skin cells it may have between about 2 mM to about 4 mM glutamine, about 1 mM ascorbic acid, about 10 mg/L insulin, about 5.5 mg/L transferrin and about 6.7 μg/L selenium.

Cell culture media for culturing corneal cells or skin cells may comprise more than about 0.2 mg/L, but less than about 20 mg/L ethanolamine. For example, it may typically comprise more than about 0.5 mg/L, but less than about 10 mg/L ethanolamine, or more than about 1 mg/L, but less than about 5 mg/L ethanolamine. Typically, cell culture media for culturing corneal cells or skin cells has about 2 mg/L ethanolamine.

The cell culture media may comprise glutamine, ascorbic acid, insulin, transferrin and/or selenium as recited above, in addition to ethanolamine.

The cell culture media may therefore comprise glutamine in the ranges cited herein, ascorbic acid at the ranges cited herein, insulin ranges cited herein, transferrin ranges cited herein selenium ranges cited herein and ethanolamine ranges cited herein. As one example, for culturing corneal cells or skin cells it may have between about 2 mM to about 4 mM glutamine, about 1 mM ascorbic acid, about 10 mg/L insulin, about 5.5 mg/L transferrin, about 6.7 μg/L selenium and about 2 mg/L ethanolamine.

Additional cell culture supplements may also be present within the cell culture medium. For example, an antibiotic such as penicillin and/or streptomycin is often present in a cell culture medium to reduce the risk of bacterial infection/contamination. Conventional concentration ranges for such antibiotics for cell culture are well known in the art and apply equally to the cell culture media described herein.

The cell culture media provided herein are particularly useful when culturing corneal cells or skin cells. As can be seen in the examples section below, the MMC agents recited herein had marked effects on corneal cell or skin cell viability and proliferation.

Specifically, the presence of PEG8, Carrageenan, PVP40, PVP360, or a combination of Ficoll® 70 and Ficoll® 400, during culture of corneal cells was shown to increase cell viability and/or proliferation. In addition, the presence of PEG35, a combination of PEG8 and PEG35; or PVP40 and PVP360 during culture of corneal cells was shown to have a beneficial effect on cell viability and/or proliferation. Similarly, the presence of PVP360, PVP40, PEG35, or a combination of Ficoll® 70 and Ficoll® 400 during the culture of skin cells was shown to increase cell viability and/or proliferation. In addition, the presence of PEG8, a combination of PEG8 and PEG35; or PVP40 and PVP360 during culture of skin cells was shown to have a beneficial effect on cell viability and/or proliferation. The use of cell culture medium comprising one or more of these specific MMCs for these cell types is therefore particularly useful when improving cell viability and/or cell proliferation is desired.

A serum-free or reduced-serum cell culture medium comprising a basal medium and one or more macromolecular crowding agents selected from the group consisting of: Carrageenan, PEG8, PEG35, PVP40 and PVP360; or a combination of PEG8 and PEG35; or Ficoll® 70 and Ficoll® 400; or PVP40 and PVP360, may therefore be used to promote cell growth, cell viability and/or proliferation of corneal cells.

In addition, a serum-free or reduced-serum cell culture medium comprising a basal medium and one or more macromolecular crowding agents selected from the group consisting of: PEG8, PEG35, PVP40, PVP360, or a combination of PEG8 and PEG35; or Ficoll® 70 and Ficoll® 400; or PVP40 and PVP360, may therefore be used to promote cell growth, cell viability and/or proliferation of skin cells.

In addition, specific MMC agents were found to increase tissue production in corneal cell culture. Specifically, the presence of PEG8, PEG35, Carrageenan, or a combination of Ficoll® 70 and Ficoll® 400, during cell culture was shown to increase tissue production by corneal cells. Furthermore, the presence of PVP40, PVP360, a combination of PEG8 and PEG35, or combination of PVP40 and PVP360 was shown to be promote tissue production by corneal cells. The use of cell culture medium comprising one or more of these specific MMCs is therefore particularly useful when tissue production is desired.

A serum-free or reduced-serum cell culture medium comprising a basal medium and one or more macromolecular crowding agents selected from the group consisting of: carrageenan, PEG8, PEG35, PVP40, PVP360, a combination of PEG8 and PEG35, a combination of PVP40 and PVP360, or a combination of Ficoll® 70 and Ficoll® 400, may therefore be used to improve tissue production from corneal cells such as corneal stromal cells.

The data described above is summarised in Tables 1 and 2 in the examples section below.

As would be clear to a person of skill in the art, any of the cell culture media described herein may be used in in vitro cell culture, particularly for the culture of corneal cells or skin cells. Uses of these cell culture media for in vitro cell culture are therefore also provided herein. The cell media compositions described in detail herein and the benefits described for specific cell types apply equally to such uses.

Methods of cell culture are also provided herein, wherein the methods comprise culturing cells in a serum-free or reduced-serum cell culture medium comprising a basal medium and one or more macromolecular crowding agents. Such methods are particularly relevant for the culture of corneal cells or skin cells. The cell media described in detail herein and the benefits described for specific cell types apply equally to such methods.

Typical cell culture methods and processes using the cell culture media described herein for the culture of corneal cells or skin cells are encompassed herein and are well known in the art. For example, the in vitro reduced-serum or serum-free cell culture method described herein may comprise culturing cells (i.e. maintaining the cells in the stated cell culture media) for a standard period of time, e.g. a minimum period of 24 hours. Conventional culturing processes may be used.

Also described herein are cell culture medium supplements. As would be clear to a person of skill in the art, these supplements are for addition to a basal medium, for example, to enable preparation of a suitable cell culture medium for use in cell culture. A cell culture supplement as defined herein therefore does not comprise basal medium.

Accordingly, a cell culture medium supplement for in vitro serum-free or reduced-serum cell culture is provided, comprising one or more macromolecular crowding agents selected from the group consisting of: PEG8, PEG35, PVP40, PVP360, Carrageenan, Ficoll® 70 and Ficoll® 400; or a combination thereof, wherein the supplement further comprises: insulin, transferrin, selenium, ethanolamine, ascorbic acid and/or glutamine. The individual components of the cell culture medium supplement are described in detail elsewhere herein, and equally here.

The terms “cell culture medium supplement” and “cell culture medium supplement formulation” are used interchangeably herein.

A cell culture supplement as defined herein is a single composition comprising a plurality of ingredients. Cell culture supplements described herein may comprise a suitable ratio of ingredients such that, when the supplement is added to a basal medium, the supplement ingredients are present in the resultant medium at their desired concentration (their working concentration). For example, the MMC agent(s), insulin, transferrin, selenium, ethanolamine, ascorbic acid and glutamine may be present in the supplement formulation at a relative ratio that enables the supplement to be added to a basal medium in an amount that each of the MMC agent(s), insulin, transferrin, selenium, ethanolamine, ascorbic acid and glutamine are present in the resultant medium at their respective working concentration. Suitable ratios of these ingredients may be determined by a person of skill in the art, using the working concentrations and concentration ranges provided herein.

For example, the cell culture supplement may comprise:
a) insulin at a concentration that when the supplement is added to a basal medium the insulin is at a final concentration of more than about 1 mg/L, but less than about 100 mg/L in the resultant cell culture medium;
b) transferrin at a concentration that when the supplement is added to a basal medium the transferrin is at a final concentration of more than about 0.5 mg/L, but less than about 10 mg/L in the resultant cell culture medium;
c) selenium at a concentration that when the supplement is added to a basal medium the selenium is at a final concentration of more than about 0.5 μg/L, but less than about 10 μg/L in the resultant cell culture medium;
d) ethanolamine at a concentration that when the supplement is added to a basal medium the ethanolamine is at a final concentration of more than about 0.2 mg/L, but less than about 20 mg/L in the resultant cell culture medium;
e) ascorbic acid at a concentration that when the supplement is added to a basal medium the ascorbic acid is at a final concentration of more than about 0.1 mM, but less than about 10 mM in the resultant cell culture medium;
f) glutamine (e.g. L-alanyl-L-glutamine dipeptide) at a concentration that when the supplement is added to a basal medium the amount of glutamine available in the medium is at a final concentration of more than about 1 mM, but less than about 10 mM in the resultant cell culture medium; and
g) a macromolecular crowding agent selected from:
   (i) PEG8 at a concentration that when the supplement is added to a basal medium the PEG8 is at a final concentration of more than about 0.25 g/L, but less than about 25 g/L in the resultant cell culture medium; or
   (ii) PEG35 at a concentration that when the supplement is added to a basal medium the PEG35 is at a final concentration of more than about 0.0 g/L, but less than about 50 g/L in the resultant cell culture medium; or
   (iii) PVP40 at a concentration that when the supplement is added to a basal medium the PVP40 is at a final concentration of more than about 0.05 g/L, but less than about 50 g/L in the resultant cell culture medium; or
   (iv) PVP360 at a concentration that when the supplement is added to a basal medium the PVP360 is at a final concentration of more than about 50 mg/L, but less than about 15 g/L in the resultant cell culture medium; or Carrageenan at a concentration that when the supplement is added to a basal medium the Carrageenan is at a final concentration of more than about 1 mg/L, but less than about 10 g/L cell in the resultant cell culture medium; or
   (v) Ficoll® 70 at a concentration that when the supplement is added to a basal medium the Ficoll® 70 is at a final concentration of more than about 300 mg/L, but less than about 300 g/L in the resultant cell culture medium; or
   (vi) Ficoll® 400 at a concentration that when the supplement is added to a basal medium the Ficoll® 400 is at a final concentration of more than about 300 mg/L, but less than about 300 g/L in the resultant cell culture medium.

In one example, the cell culture supplement comprises (a) to (f), in addition to (g)(i). For example, when culturing corneal cells or skin cells, the cell culture supplement may comprise (a) to (f), and PEG8 at a concentration that when the supplement is added to a basal medium the PEG8 is at a final concentration of more than about 0.25 g/L, but less than about 25 g/L in the resultant cell culture medium. For example, it may comprise (a) to (f) and PEG8 at a concentration that when the supplement is added to a basal medium the PEG8 is at a final concentration of more than about 0.5 g/L, but less than about 10 g/L e.g. more than about 0.5 g/L, but less than about 5 g/L. Typically, it may comprise (a) to (f) and PEG8 at a concentration that when the supplement is added to a basal medium the PEG8 is at a final concentration of about 1.1 g/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and PEG8 at a concentration that when the supplement is added to a basal medium the PEG8 is at a final concentration of about 1.1 g/L.

In another example, the cell culture supplement comprises (a) to (f), in addition to (g)(ii). For example, when culturing corneal cells or skin cells, the cell culture supplement may comprise (a) to (f), and PEG35 at a concentration that when the supplement is added to a basal medium the PEG35 is at a final concentration of more than about 0.5 g/L, but less than about 50 g/L in the resultant cell culture medium. For example, it may comprise (a) to (f) and PEG35 at a concentration that when the supplement is added to a basal medium the PEG35 is at a final concentration of more than about 0.5 g/L, but less than about 25 g/L e.g. more than about 1 g/L, but less than about 10 g/L. Typically, it may comprise (a) to (f) and PEG35 at a concentration that when the supplement is added to a basal medium the PEG35 is at a final concentration of about 2 g/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and PEG35 at a concentration that when the supplement is added to a basal medium the PEG35 is at a final concentration of about 2 g/L.

In another example, the cell culture supplement comprises (a) to (f), in addition to (g)(iii). For example, when culturing corneal cells or skin cells, the cell culture supplement may comprise (a) to (f), and PVP40 at a concentration that when the supplement is added to a basal medium the PVP40 is at a final concentration of more than about 0.05 g/L, but less than about 50 g/L in the resultant cell culture medium. For example, it may comprise (a) to (f) and PVP40 at a concentration that when the supplement is added to a basal medium the PVP40 is at a final concentration of more than about 0.5 g/L, but less than about 25 g/L e.g. more than about 1 g/L, but less than about 10 g/L. Typically, it may comprise (a) to (f) and PVP40 at a concentration that when the supplement is added to a basal medium the PVP40 is at a final concentration of about 4.5 g/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and PVP40 at a concentration that when the supplement is added to a basal medium the PVP40 is at a final concentration of about 4.5 g/L.

In another example, the cell culture supplement comprises (a) to (f), in addition to (g)(iv). For example, when culturing corneal cells or skin cells, the cell culture supplement may comprise (a) to (f), and PVP360 at a concentration that when the supplement is added to a basal medium the PVP360 is at a final concentration of more than about 50 mg/L, but less than about 15 g/L in the resultant cell culture medium. For example, it may comprise (a) to (f) and PVP360 at a concentration that when the supplement is added to a basal medium the PVP360 is at a final concentration of more than about 0.5 g/L, but less than about 15 g/L e.g. more than about 1 g/L, but less than about 15 g/L. Typically, it may comprise (a) to (f) and PVP360 at a concentration that when the supplement is added to a basal medium the PVP360 is at a final concentration of about 10 g/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and PVP360 at a concentration that when the supplement is added to a basal medium the PVP360 is at a final concentration of about 10 g/L.

In one example, the cell culture supplement comprises (a) to (f), in addition to (g)(v). For example, when culturing corneal cells or skin cells, the cell culture supplement may comprise (a) to (f), and carrageenan at a concentration that when the supplement is added to a basal medium the carrageenan is at a final concentration of more than about 1 mg/L, but less than about 10 g/L in the resultant cell culture medium. For example, it may comprise (a) to (f) and carrageenan at a concentration that when the supplement is added to a basal medium the carrageenan is at a final concentration of more than about 1 mg/L, but less than about 1 g/L e.g. more than about 5 mg/L, but less than about 100 mg/L in the resultant cell culture medium. Typically, it may comprise (a) to (f) and carrageenan at a concentration that when the supplement is added to a basal medium the carrageenan is at a final concentration of about 10 mg/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and carrageenan at a concentration that when the supplement is added to a basal medium the carrageenan is at a final concentration of about 10 mg/L.

In another example, the cell culture supplement comprises (a) to (f), in addition to (g)(vi). For example, when culturing corneal cells or skin cells, the cell culture supplement may comprise (a) to (f), and Ficoll® 70 at a concentration that when the supplement is added to a basal medium the Ficoll® 70 is at a final concentration of more than about 300 mg/L, but less than about 300 g/L in the resultant cell culture medium. For example, it may comprise (a) to (f) and Ficoll® 70 at a concentration that when the supplement is added to a basal medium the Ficoll® 70 is at a final concentration of more than about 500 mg/L, but less than about 100 g/L e.g. more than about 1 g/L, but less than about 50 g/L. Typically, it may comprise (a) to (f) and Ficoll® 70 at a concentration that when the supplement is added to a basal medium the Ficoll® 70 is at a final concentration of about 10 g/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and Ficoll® 70 at a concentration that when the supplement is added to a basal medium the Ficoll® 70 is at a final concentration of about 10 g/L.

In another example, the cell culture supplement comprises (a) to (f), in addition to (g)(vii). For example, when culturing corneal cells or skin cells, the cell culture supplement may comprise (a) to (f), and Ficoll® 400 at a concentration that when the supplement is added to a basal medium the Ficoll® 400 is at a final concentration of more than about 300 mg/L, but less than about 300 g/L in the resultant cell culture medium. For example, it may comprise (a) to (f) and Ficoll® 400 at a concentration that when the supplement is added to a basal medium the Ficoll® 400 is at a final concentration of more than about 375 mg/L, but less than about 75 g/L e.g. more than about 1 g/L, but less than about 50 g/L. Typically, it may comprise (a) to (f) and Ficoll® 400 at a concentration that when the supplement is added to a basal medium the Ficoll® 400 is at a final concentration of about 7.5 g/L.

In one example, the cell culture supplement comprises insulin at a concentration that when the supplement is added to a basal medium the insulin is at a final concentration of about 10 mg/L; transferrin at a concentration that when the supplement is added to a basal medium the transferrin is at a final concentration of about 5.5 mg/L; selenium at a concentration that when the supplement is added to a basal medium the selenium is at a final concentration of about 6.7 μg/L; ethanolamine at a concentration that when the supplement is added to a basal medium the ethanolamine is at a final concentration of about 2 mg/L; ascorbic acid at a concentration that when the supplement is added to a basal medium the ascorbic acid is at a final concentration of about 1 mM; L-alanyl-L-glutamine dipeptide at a concentration that when the supplement is added to a basal medium the L-alanyl-L-glutamine dipeptide is at a final concentration of about 4 mM; and Ficoll® 400 at a concentration that when the supplement is added to a basal medium the Ficoll® 400 is at a final concentration of about 7.5 g/L.

Optimal final concentration ranges and values for each of these ingredients is provided elsewhere herein. These apply equally to the ranges, values and combinations that may be used in the cell culture supplements provided herein. Suitable ratios of the ingredients in the supplement can readily be derived by a person of skill in the art based on the disclosure here.

Cell culture supplements described herein are typically formulated at as a concentrated supplement formulation, which can be appropriately diluted e.g. in basal medium for use. In this context, the concentrated supplement may be of any suitable concentration e.g. it may be a 5× formulation, a 10× formulation, a 50× formulation etc. In this context, a 1× formulation represents the working concentration of ingredients in the supplement (i.e. the concentration of these ingredients that is needed when they are present in the cell culture medium). In other words, a 1× formulation represents the “working concentration” of the ingredients. The working concentration is also referred to as a “final concentration” herein.

The term “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture medium used for culturing cells in vitro. A cell culture medium used for the in vitro culture of cells is a 1× formulation by definition.

When a number of ingredients are present, each ingredient in a 1× formulation has a concentration about equal to the concentration of each respective ingredient in a medium during cell culturing. For example, RPMI-1640 culture medium contains, among other ingredients, 0.2 g/L L-arginine, 0.05 g/L L-asparagine, and 0.02 g/L L-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See, for example, Methods For Preparation of Media, Supplements and Substrate For Serum-Free Animal Cell Culture Allen R. Liss, N.Y. (1984), Handbook of Microbiological Media, Second Ed., Ronald M. Atlas, ed. Lawrence C. Parks (1997) CRC Press, Boca Raton, FL and Plant Culture Media, Vol. 1: Formulations and Uses E. F. George, D. J. M. Puttock, and H. J. George (1987) Exegetics Ltd. Edington, Westbury, Wilts, BA13 4QG England. The osmolarity and/or pH, however, can differ in a 1× formulation compared to the culture medium, particularly when fewer ingredients are contained in the 1× formulation.

A “10× formulation” is meant to refer to a solution wherein the concentration of each ingredient in that solution is about 10 times more than the concentration of each respective ingredient in a medium during cell culturing. For example, a 10× formulation of RPMI-1640 culture medium can contain, among other ingredients, 2.0 g/L L-arginine, 0.5 g/L L-asparagine, and 0.2 g/L L-aspartic acid (compare 1× formulation, above). A “10× formulation” can contain a number of additional ingredients at a concentration about 10 times that found in the 1× culture formulation. As will be readily apparent, “25× formulation,” “50× formulation,” “100× formulation,” “500× formulation,” and “1000× formulation” designate solutions that contain ingredients at about 25-, 50-, 100-, 500-, or 1000-fold concentrations, respectively, as compared to a 1× cell culture formulation. Again, the osmolarity and pH of the medium formulation and concentrated solution can vary.

The supplement formulations described herein may be suitably concentrated as, for example, a 10×, 20×, 25×, 50×, 100×, 500×, or 1000× supplement formulation.

A particularly preferred example is a 50× supplement formulation. In this context, the supplement formulation may be a 50× concentrated liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and a macromolecular crowding agent selected from the group consisting of: 55 g/L PEG8, 225 g/L PVP40, 100 g/L PEG35, 500 g/L PVP360, 0.5 g/L Carrageenan, and 50 g/L Ficoll® 70 and 37.5 g/L Ficoll® 400.

For example, the supplement formulation may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 55 g/L PEG8.

In a further example, the supplement formulation may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 225 g/L PVP40.

Alternatively, the supplement formulation may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 100 g/L PEG35.

Furthermore, the supplement formulation may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 500 g/L PVP360.

Alternatively, the supplement formulation may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 0.5 g/L Carrageenan.

Furthermore, the supplement formulation may be a 50× concentrate liquid solution, and the liquid solution may comprise: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 50 g/L Ficoll® 70 and 37.5 g/L Ficoll® 400.

In other words, the supplement formulation may be a 50× concentrate liquid solution, and the liquid solution may comprise: insulin, transferrin, selenium, ethanolamine, ascorbic acid and a macromolecular crowding agent selected from the group consisting of: PEG8, PVP40, PEG35, PVP360, Carrageenan, Ficoll® 70 and Ficoll® 400, wherein the insulin, transferrin, selenium, ethanolamine, ascorbic acid and MMC agent consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and a macromolecular crowding agent selected from the group consisting of: 55 g/L PEG8, 225 g/L PVP40, 100 g/L PEG35, 500 g/L PVP360, 0.5 g/L Carrageenan, and 50 g/L Ficoll® 70 and 37.5 g/L Ficoll® 400.

For example, the supplement formulation may be a 50× concentrate liquid solution, and the liquid solution may comprise: insulin, transferrin, selenium, ethanolamine, ascorbic acid and PEG8, wherein the insulin, transferrin, selenium, ethanolamine, ascorbic acid and PEG8 consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 55 g/L PEG8.

In a further example, the supplement formulation may be a 50× concentrate liquid solution, and the liquid solution may comprise: insulin, transferrin, selenium, ethanolamine, ascorbic acid and PVP40, wherein the insulin, transferrin, selenium, ethanolamine, ascorbic acid and PVP40 consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 225 g/L PVP40.

Alternatively, the supplement formulation may be a 50× concentrate liquid solution, and the liquid solution may comprise: insulin, transferrin, selenium, ethanolamine, ascorbic acid and PEG35, wherein the insulin, transferrin, selenium, ethanolamine, ascorbic acid and PEG35 consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 100 g/L PEG35.

Furthermore, the supplement formulation may be a 50× concentrate liquid solution, and the liquid solution may comprise: insulin, transferrin, selenium, ethanolamine, ascorbic acid and PVP360, wherein the insulin, transferrin, selenium, ethanolamine, ascorbic acid and PVP360 consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 500 g/L PVP360.

Alternatively, the supplement formulation may be a 50× concentrate liquid solution, and the liquid solution may comprise: insulin, transferrin, selenium, ethanolamine, ascorbic acid and Carrageenan, wherein the insulin, transferrin, selenium, ethanolamine, ascorbic acid and Carrageenan consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 0.5 g/L Carrageenan.

Furthermore, the supplement formulation may be a 50× concentrate liquid solution, and the liquid solution may comprise: insulin, transferrin, selenium, ethanolamine, ascorbic acid and Ficoll® 70 and Ficoll® 400, wherein the insulin, transferrin, selenium, ethanolamine, ascorbic acid and Ficoll® 70 and Ficoll® 400 consist of: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and 50 g/L Ficoll® 70 and 37.5 g/L Ficoll® 400.

As would be clear to a person of skill in art, the supplement can be prepared in different forms, such as dry powder media (“DPM”), a granulated preparation (which requires addition of water, but not other processing, such as pHing), liquid media or as media concentrates. The supplement may therefore be a liquid solution or a dry powder or a granulated dry powder.

A hermetically-sealed vessel containing a serum-free or reduced-serum cell culture medium or a cell culture medium supplement described herein is also provided.

By “vessel” is meant any container, for example, a glass, plastic, or metal container, that can provide an aseptic environment for storing a serum-free or reduced-serum cell culture medium or a cell culture medium supplement as described here. The vessel may have any volume, for example it may suitably be a vessel that is configured to hold about 500 ml or about 1 L of serum-free or reduced-serum cell culture medium or a cell culture medium supplement.

A hermetic seal is any type of sealing that makes a given object airtight (preventing the passage of air, oxygen, or other gases). The term originally applied to airtight glass containers, but as technology advanced it applied to a larger category of materials, including rubber and plastics.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Examples

### 1.1 Testing Polyethylene Glycol 8 kDa (PEG 8) on Corneal Cells

### Experimental Methods

Corneal stromal cells isolated from human donor corneas were grown with 2 different serum-free culture media: SFM* control, comprising DMEM-low glucose (2 g/L) with 1×ITS liquid medium, 1 mM ascorbic acid, 10 μM retinoic acid, and 1% antibiotic supplements; +PEG, same as SFM* plus 20 g/L PEG8; and +PEG+PA, same as SFM* plus 20 g/L PEG 8 and 1 μm ETTES-PA. These formulations were tested for their capacity to promote accelerated tissue formation in serum-free conditions.

Changes in proliferation were determined after 2, 4, and 6 weeks of incubation using AlamarBlue™ viability assay (Thermo Fisher Scientific). Tissue formation was evaluated after 6 weeks in culture, analysed in terms of thickness, weight, hydration, and composition fluorescence microscopy, and quantified using the ImageJ software.

### Proliferation—PEG8 Increases Cell Proliferation of Human Corneal Stromal Cells

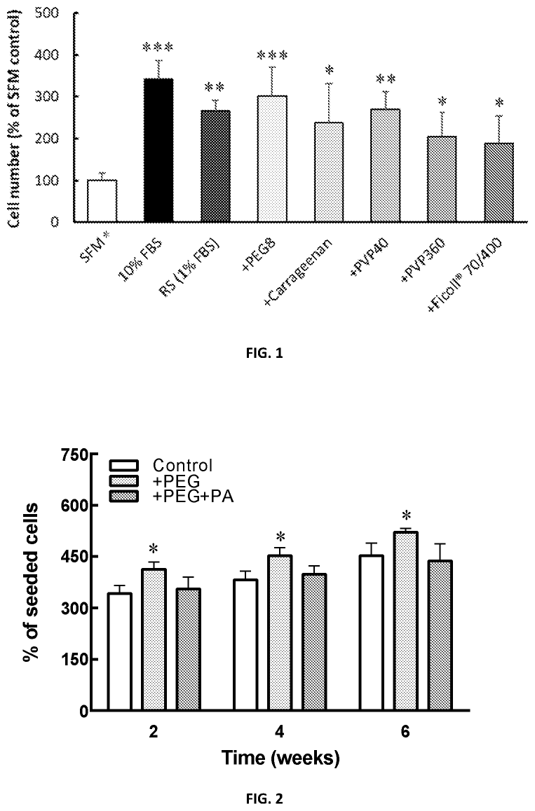

It was observed +PEG supplementation significantly increased cell numbers over all time points tested, but +PEG+PA did not. See FIG. 1.

### Tissue Formation—PEG 8 Promotes Formation of Corneal Stromal Tissue

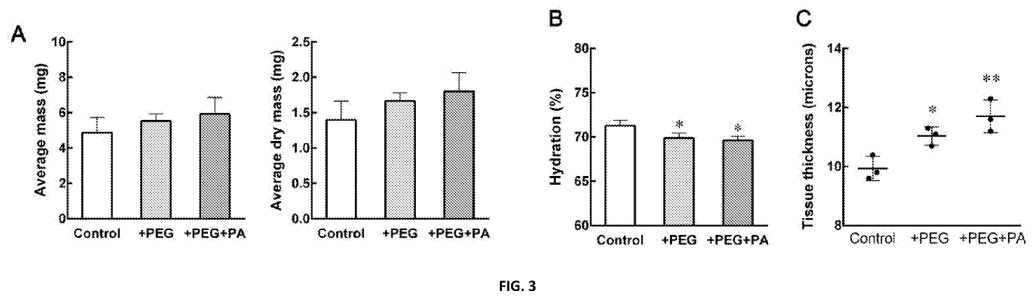

After a period of 6 weeks in culture, tissues formed by cells grown with different media were analysed and compared in terms of thickness, weight and hydration (FIG. 2), as well as specific corneal cell and matrix components (FIG. 3). Total biomass was increased (albeit not significantly) in +PEG and +PEG+PA, with these two conditions resulting in significantly thicker, less hydrated (denser) tissues (FIG. 2). Moreover, MMC-supplemented media increased deposition of corneal-specific ECM components, namely collagen I and V and keratocan (FIG. 3).

### Conclusions

PEG8 treatment increases corneal stromal cell proliferation
PEG8 treatment with 20 g/l results in improved tissue bio-fabrication, with increased deposition of corneal-specific ECM

### 2.1 Effects of MMC Supplements on Skin Fibroblast Proliferation

Fish skin fibroblasts were seeded at 1.5×10³ cells/cm² in 24 well plates in DMEM/F12 medium supplemented with 1% Penicillin/Streptomycin (SFM). To allow for cells attachment, cultures were incubated in a humidified atmosphere at 37° C. and 5% (v/v) CO₂ for 4 hours. Culture media was then exchanged with medium supplemented with MMC agents or with 1% FBS, and cells grown for 3 days. Cell numbers were presented as percentage of SFM control and statistical analysis was performed using a two-way ANOVA with differences between treatments and untreated control using Dunnett multiple comparisons test.

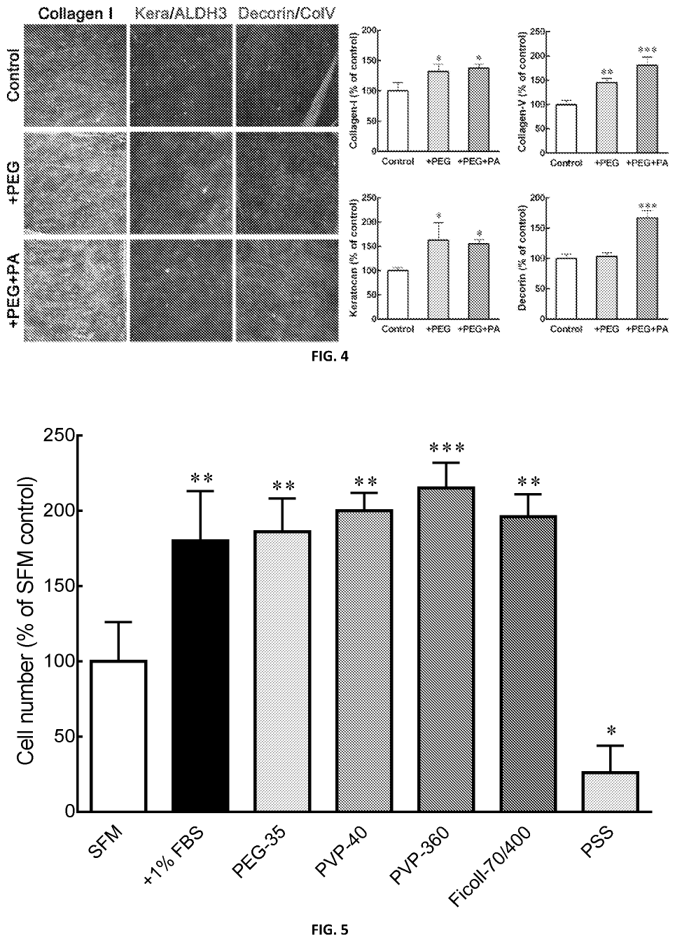

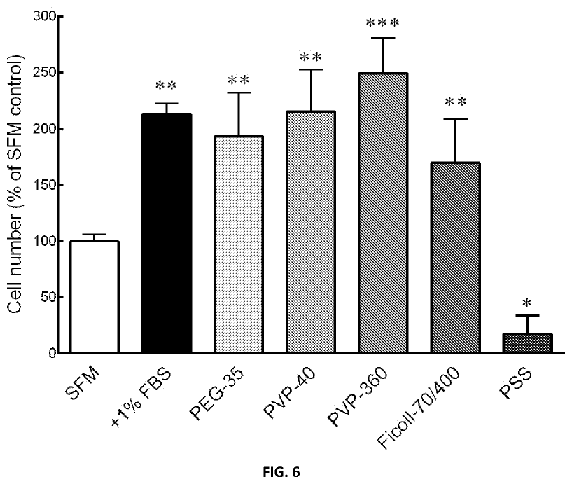

Conclusions: PEG35, PVP40, and PVP360 promoted cell growth, whilst PSS was deleterious. Lambda Carrageenan inhibited fibroblast proliferation. PEG8 had marginal effect (data not shown). Finally, Ficoll® 70/400 was shown to promote fibroblast proliferation. See FIG. 4.

A summary of the data provided herein is below.

TABLE 1
the effect of different MMC agents on the proliferation and tissue
production of corneal stromal cells. The MMC agents are ranked
in order of effect (with 1 being the most beneficial).

| Proliferation | Tissue production |
|---|---|
| Promotors | Promotors |
| 1. PEG8 | 1. PEG8 |
| 2. Lambda Carrageenan | 2. PEG35 |
| 3. PVP40 | 3. Ficoll70:400 mix |
| 4. PVP360 | 4. Lambda Carrageenan |
| 5. Ficoll70:400 mix | Promotors (weak) |
| Promotors (weak) | 5. PVP40 |
| 6. PEG35 | 6. PEG8:35 mix |
| 7. PEG8:35 mix | 7. PVP360 |
| 8. PVP40:360 mix | 8. PVP40:360 mix |
| Toxic | Toxic |
| 9. DxS40 | 9. DxS40 |
| 10. Dx500 | 10. Dx500 |
| 11. DxS40:500 mix | 11. DxS40:500 mix |
| 12. PSS | 12. PSS |

TABLE 2
the effect of different MMC agents on the proliferation of skin
cells. The MMC agents are ranked in order of effect (with 1
being the most beneficial).
Proliferation

Promotors
1. PVP360
2. PVP40
3. Ficoll70:400 mix
4. PEG35
Promotors (weak)
5. PEG8
6. PEG8:35 mix
7. PVP40:360 mix
Toxic
8. Lambda Carrageenan
9. DxS40
10. Dx500
11. PSS
12. DxS40:500 mix

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

1. De Pieri, A., et al., Seaweed polysaccharides as macromolecular crowding agents. Int J Biol Macromol, 2020. 164: p. 434-446.
2. Gouveia, R. M., et al., Template Curvature Influences Cell Alignment to Create Improved Human Corneal Tissue Equivalents. Adv Biosyst, 2017. 1(12): p. e1700135.
3. Gouveia, R. M., et al., Assessment of corneal substrate biomechanics and its effect on epithelial stem cell maintenance and differentiation. Nat Commun, 2019. 10(1): p. 1496.
4. Jia, M., et al., Effect of polyethylene glycol as a molecular crowding agent on reducing template consumption for preparation of molecularly imprinted polymers. Analytical Methods, 2016. 8(23): p. 4554-4562.
5. Kuznetsova, I. M., K. K. Turoverov, and V. N. Uversky, What Macromolecular Crowding Can Do to a Protein. International Journal of Molecular Sciences, 2014. 15(12): p. 23090-23140.
6. Bharadwaj, S., et al., Higher molecular weight polyethylene glycol increases cell proliferation while improving barrier function in an in vitro colon cancer model. J Biomed Biotechnol, 2011. 2011: p. 587470.
7. Patrikoski, M., et al., Effects of Macromolecular Crowding on Human Adipose Stem Cell Culture in Fetal Bovine Serum, Human Serum, and Defined Xeno-Free/Serum-Free Conditions. Stem Cells International, 2017. 2017: p. 6909163.
8. Benny, P. and M. Raghunath, Making microenvironments: A look into incorporating macromolecular crowding into in vitro experiments, to generate biomimetic microenvironments which are capable of directing cell function for tissue engineering applications. J Tissue Eng, 2017.8: p. 2041731417730467.
9. Haaf, F., A. Sanner, and F. Straub, Polymers of N-Vinylpyrrolidone: Synthesis, Characterization and Uses. Polymer Journal, 1985. 17(1): p. 143-152.
10. Rashid, R., et al., Novel use for polyvinylpyrrolidone as a macromolecular crowder for enhanced extracellular matrix deposition and cell proliferation. Tissue Eng Part C Methods, 2014. 20(12): p. 994-1002.

1. Use of a serum-free or reduced-serum cell culture medium comprising a basal medium and one or more macromolecular crowding agents for in vitro cell culture to promote cell proliferation, wherein:
a) the cell is a skin cell and the macromolecular crowding agent is selected from the group consisting of: PEG8, PEG35, PVP40, and PVP360; or a combination thereof; or
b) the cell is a corneal cell and the macromolecular crowding agent is selected from the group consisting of: PEG8, PVP40, PVP360, and PEG35; or a combination thereof.

2. An in vitro serum-free or reduced-serum cell culture method to promote cell proliferation, comprising culturing cells in a serum-free or reduced-serum cell culture medium comprising a basal medium and one or more macromolecular crowding agents, wherein:
a) the cells are skin cells and the macromolecular crowding agent is selected from the group consisting of: PEG8, PEG35, PVP40, PVP360; or a combination thereof; or
b) the cells are corneal cells and the macromolecular crowding agent is selected from the group consisting of: PEG8, PVP40, PVP360, PEG35; or a combination thereof.

3. The use or method according to claim 1 or claim 2, wherein the cells are skin cells and the macromolecular crowding agent is a combination of: PEG8 and PEG35; or PVP40 and PVP360.

4. The use or method according to claim 1 or claim 2, wherein the cells are corneal cells and the macromolecular crowding agent is a combination of: PEG8 and PEG35; PVP40 and PVP360.

5. A serum-free or reduced-serum cell culture medium for in vitro cell culture, wherein the cell culture medium comprises a basal medium and one or more macromolecular crowding agents selected from the group consisting of: PEG8, PVP40, PVP360 and PEG35; or a combination thereof.

6. The cell culture medium of claim 5, wherein the macromolecular crowding agent is a combination of: PVP40 and PVP360; or PEG8 and PEG35.

7. The cell culture medium, use or method according to any preceding claim, wherein the basal medium is DMEM/F12.

8. The cell culture medium, use or method according to any preceding claim, wherein the cell culture medium is a serum-free cell culture medium, optionally wherein the cell culture medium does not contain materials obtained from an animal.

9. The cell culture medium, use or method according to claim 8, wherein the serum-free cell culture medium is a chemically defined cell culture medium.

10. The cell culture medium, use or method according to any preceding claim, wherein the cell culture medium further comprises glutamine, optionally wherein the cell culture medium further comprises ascorbic acid, insulin, transferrin, selenium, and ethanolamine.

11. The cell culture medium, use or method according to claim 10, wherein the cell culture medium comprises more than about 1 mM, but less than about 10 mM L-alanyl-L-glutamine dipeptide; and optionally:
a) more than about 0.1 mM, but less than about 10 mM ascorbic acid; and
b) more than about 1 mg/L, but less than about 100 mg/L insulin; and
c) more than about 0.5 mg/L, but less than about 10 mg/L transferrin; and
d) more than about 0.5 μg/L, but less than about 10 μg/L selenium; and
e) more than about 0.2 mg/L, but less than about 20 mg/L ethanolamine.

12. The cell culture medium, use or method according to any preceding claim, wherein the cell culture medium further comprises penicillin and streptomycin.

13. A cell culture medium supplement for in vitro serum-free or reduced-serum cell culture, comprising one or more macromolecular crowding agents selected from the group consisting of: PVP40, PEG8, PVP360 and PEG35; or a combination thereof, wherein the supplement further comprises: insulin, transferrin, selenium, ethanolamine, ascorbic acid and/or glutamine.

14. The supplement of claim 13, comprising:
a) insulin at a concentration that when the supplement is added to a basal medium the insulin is at a final concentration of more than about 1 mg/L, but less than about 100 mg/L in the resultant cell culture medium;
b) transferrin at a concentration that when the supplement is added to a basal medium the transferrin is at a final concentration of more than about 0.5 mg/L, but less than about 10 mg/L in the resultant cell culture medium;
c) selenium at a concentration that when the supplement is added to a basal medium the selenium is at a final concentration of more than about 0.5 μg/L, but less than about 10 μg/L in the resultant cell culture medium;
d) ethanolamine at a concentration that when the supplement is added to a basal medium the ethanolamine is at a final concentration of more than about 0.2 mg/L, but less than about 20 mg/L in the resultant cell culture medium;
e) ascorbic acid at a concentration that when the supplement is added to a basal medium the ascorbic acid is at a final concentration of more than about 0.1 mM, but less than about 10 mM in the resultant cell culture medium;
f) L-alanyl-L-glutamine dipeptide at a concentration that when the supplement is added to a basal medium the amount of glutamine available in the medium is at a final concentration of more than about 1 mM, but less than about 10 mM in the resultant cell culture medium; and
g) a macromolecular crowding agent selected from:
(i) PEG8 at a concentration that when the supplement is added to a basal medium the PEG8 is at a final concentration of more than about 0.25 g/L, but less than about 25 g/L in the resultant cell culture medium; or
(ii) PEG35 at a concentration that when the supplement is added to a basal medium the PEG35 is at a final concentration of more than about 0.5 g/L, but less than about 50 g/L in the resultant cell culture medium; or
(iii) PVP40 at a concentration that when the supplement is added to a basal medium the PVP40 is at a final concentration of more than about 0.05 g/L, but less than about 50 g/L in the resultant cell culture medium; or
(iv) PVP360 at a concentration that when the supplement is added to a basal medium the PVP360 is at a final concentration of more than about 50 mg/L, but less than about 15 g/L in the resultant cell culture medium.

15. The supplement of claim 14, comprising:
a) insulin at a concentration that when the supplement is added to a basal medium the insulin is at a final concentration of about 10 mg/L;
b) transferrin at a concentration that when the supplement is added to a basal medium the transferrin is at a final concentration of about 5.5 mg/L;
c) selenium at a concentration that when the supplement is added to a basal medium the selenium is at a final concentration of about 6.7 μg/L;
d) ethanolamine at a concentration that when the supplement is added to a basal medium the ethanolamine is at a final concentration of about 2 mg/L;
e) ascorbic acid at a concentration that when the supplement is added to a basal medium the ascorbic acid is at a final concentration of about 1 mM;
f) L-alanyl-L-glutamine dipeptide at a concentration that when the supplement is added to a basal medium the L-alanyl-L-glutamine dipeptide is at a final concentration of about 4 mM; and
g) a macromolecular crowding agent selected from the group consisting of: PEG8 at a concentration that when the supplement is added to a basal medium the PEG8 is at a final concentration of about 1.1 g/L; PEG35 at a concentration that when the supplement is added to a basal medium the PEG35 is at a final concentration of about 2 g/L; PVP40 at a concentration that when the supplement is added to a basal medium the PVP40 is at a final concentration of about 4.5 g/L; and PVP360 at a concentration that when the supplement is added to a basal medium the PVP360 is at a final concentration of about 10 g/L.

16. The supplement of any of claims 13 to 15, wherein the supplement is a liquid solution or a dry powder or a granulated dry powder.

17. The supplement of claim 16, wherein the supplement is a 50× concentrate liquid solution, and the liquid solution comprises: 0.5 g/L insulin, 0.27 g/L transferrin, 0.35 ml/L selenium, 0.1 g/L ethanolamine, 50 mM ascorbic acid, 100 mM L-alanyl-L-glutamine dipeptide and a macromolecular crowding agent selected from the group consisting of: 55 g/L PEG8, 225 g/L PVP40, 100 g/L PEG35, and 500 g/L PVP360.

18. A hermetically-sealed vessel containing a serum-free or reduced-serum cell culture medium or a cell culture medium supplement according to any of the preceding claims.
